(19) United States
(12) Patent Application Publication
(10) Pub. No.: US 2003/0143744 A1
(43) Pub. Date: Jul. 31, 2003

(54) Method for the production of cotton somatic embryos

(76) Inventors: James Martin Dunwell, Berkshire (GB); Deborah Jane Keith, Berkshire (GB); Jose Manso Preto Nobre, Apartado (PT)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/220,837

(22) PCT Filed: Mar. 12, 2001
(86) PCT No.: PCT/GB01/01071

(30) Foreign Application Priority Data
Mar. 15, 2000 (GB) 0006247.1

(51) Int. Cl. C12N 5/04; C12N 15/82

(52) U.S. Cl. 435/468; 435/419

(57) ABSTRACT

The present invention provides Inter alia, a method for the production of cotton somatic embryos comprising (a) isolating a totipotent stomatal cell-containing epidermal explant from leaf material excised from a cotton plant; and (b) culturing said explant in a basal medium which comprises an embryogenic callus-inducing quantity of an auxin and a cytokinin under an embryogenic callus inducing intensity of light until embryogenic callus is formed; and (c) sub-culturing said embryogenic callus onto a somatic embryo differentiation media to produce said somatic embryos. Plants may be regenerated from the somatic embryos and in a particular embodiment of the invention said totipotent stomatal cell is transformed, prior to the inducement of embryogenic callus, with a polynucleotide that provides for a desired agronomic trait.

The present invention relates inter alia to a method for the production of cotton somatic embryos. More specifically the invention relates to a method for the production of cotton somatic embryos and the regeneration of cotton plants therefrom wherein said somatic embryos are produced from callus material which is produced from totipotent stomatal cell-containing epidermal explants. Preferably the stomatal cells are transformed with exogenous DNA prior to the production of the somatic embryos and the subsequent plant regeneration.

Cotton (Gossypium hirsutum L.) is the most important textile crop economically and the world's second most important oilseed crop after soybean. It is cultivated and grown in a variety of areas world wide, mainly in subtropical and tropical environmental conditions. Cotton is grown for the production of spinnable fibers and seed products such as oil, meal and seed hulls and in addition, short fibers called linters are removed from cotton seed and used in cellulose production. For this reason, there has long been interest in breeding such an economically important crop species.

As well as conventional breeding, biotechnological approaches including the development of tissue culture and transformation procedures are in use for cotton breeding. New transgenic varieties containing bacterial genes encoding herbicide resistance and the Bt endotoxin have been recently released. In addition, stress resistance and fiber improvement are major targets for cotton improvement.

Guard cells, which are situated in the epidermal tissue and part of the stomatal complex, have unique functional properties involving the interaction between a plant and its environment. These interactions include the modulation of light penetration, gas exchange for photosynthesis and water supply.

Epidermal strips have long been used as tools for the study of stomatal mechanisms, however, nowadays, the availability of efficient procedures to isolate guard cells and to develop regeneration systems are basic techniques required for the application of molecular genetic approaches to stomatal biology and gene function. In addition, plant cellular differentiation and stomatal physiology associated with the expression of guard cell specific genes and pathways, may be studied using guard cells as models. Despite their high degree of functional differentiation, the totipotency of guard cell protoplasts was demonstrated recently in tobacco and sugar beet.

A large number of cultivars of cotton are still difficult to regenerate in vitro via somatic embryogenesis and therefore, plant regeneration remains genotype-dependent.

The present invention therefore seeks to provide inter alia, a procedure for the production of somatic embryos from totipotent stomatal-cell-containing explants from cotton plants and further, the regeneration of cotton plants from said embryos. Such explants may avoid a lengthy protoplast isolation procedure used in previous studies and permit a study of the factors affecting stomatal cell dedifferentiation and regeneration in vitro.

According to the present invention there is provided a method for the production of cotton somatic embryos comprising (a) isolating a totipotent stomatal cell-containing epidermal explant from leaf material excised from a cotton plant; and (b) culturing said explant in a basal medium which comprises an embryogenic callus-inducing quantity of an auxin and cytokinin under an embryogenic callus inducing intensity of light until embryogenic callus is formed; and (c) sub-culturing said embryogenic callus onto a somatic embryo differentiation media to produce said somatic embryos. In a further embodiment of the invention the leaf material used is obtained from a flowering cotton plant. In a still further embodiment of the invention the leaf material is excised from an area attached to or surrounding an opening flower of a cotton plant. In a still further embodiment of the invention the leaf material is excised when flower opening is just initiated. In a still further embodiment of the invention the leaf material is excised when the petals of the flower start to become visible. In a still further embodiment of the present invention the leaf material used in accordance with the methods described in this specification may be obtained from a cotton plant that is at a development stage substantially similar to the one shown in as stage “B” in FIG. 1 of Nobre et al (2001), Plant Cell Reports. Page 9.

In a further embodiment of the present invention said auxin is naphthalene acetic acid (NAA) and/or said cytokinin is isopentyladenine (iP).

In a further embodiment of the present invention said stomatal cell comprises a guard cell. The person skilled in the art will recognise that explants used in the methods of the present invention may be maintained in culture to provide a readily available source of totipotent stomatal cells. Alternatively, such explants may be obtained directly from leaf material of a cotton plant when required in a manner as described below.

In a further embodiment of the invention the basal medium comprises between about 2 to about 22 μM of NAA and between about 1 to about 5 μM of iP under light irradiation of less than 21 μmol.m⁻².s⁻¹. In a still further embodiment of the invention said basal medium comprises about 10.7 μM NAA and about 4.9 μM iP. In a still further embodiment of the present invention the basal medium comprises about 10.7 to about 21.4 μM NAA and about 1.3 μM iP and the said embryogenic callus is further sub-cultured onto a basal medium comprising about 10.7 μM NAA and about 4.9 μM iP prior to sub-culturing according to step (c) as recited above.

The present invention still further provides a method as described above wherein the light irradiation is about 15.8 μmol.m⁻².s⁻¹. In a further embodiment of the invention said light irradiation is less than 15.81 μmol.m⁻².s⁻¹.

The present invention still further provides a method as described above wherein said leaf material comprises a bract or bracteole. The person skilled in the art can easily identify these bracts or bracteoles. In a further embodiment of the invention said leaf material may comprise a young leaf. In a still further embodiment of the invention said material may comprise the base region, top region or the whole of the bract or bracteole. In a still further embodiment of the invention said material comprises an epidermal strip or an epidermal region.

The present invention still further provides a method as described above wherein said explant is obtained from a cotton plant that is between about 4 to about 10 months old. In a further embodiment of the invention said explant is obtained from a cotton plant that is between about 4 to about 5 months old. In a still further embodiment of the invention said explant is obtained from a cotton plant that is between about 9 to about 10 months old.

The present invention still further provides a method as described above wherein said leaf material is sterilised prior to production of the said explant. In a still further embodiment of the invention said explant comprises an epidermal strip or an epidermal region.

The present invention still further provides a method as described above wherein said explant is orientated such that the cuticle of said explant is in contact with said medium.

The present invention further provides a method as described above wherein said somatic embryo differentiation media comprises about 0.15 to about 0.4 μM of abscisic acid (ABA). In a further embodiment of the invention said somatic embryo differentiation media comprises about 0.19 to about 0.38 μM of ABA.

The present invention further provides a method as described above wherein the said cell is transformed with a polynucleotide prior to induction of embryogenic callus. In a further embodiment of the invention said polynucleotide provides for the production of an agronomic trait selected from the group consisting of: herbicide resistance; insect resistance; nematode resistance; fungal resistance; viral resistance; stress tolerance; altered yield; fibre quality and oil quality. In a still further embodiment of the invention said polynucleotide provides for the production of a 5-enolpyruvylshikimate-3-phosphate synthase and/or a crystal endotoxin protein (CRY) and/or a vegetative insecticidal protein (VIP) or the polynucleotide provides for resistance to a herbicide selected from the group consisting of: glyphosate; paraquat; acifluorfen; chlorimuron-ethyl; fomesafen; acetochlor; fluazifop-P-butyl; and metolachlor. In a still further embodiment of the present invention the polynucleotide provides for resistance to insect pests including Lepidoptera, Spodoptera, Coleoptera, Diptera, Hemiptera, Homoptera, Thysonoptera and/or nematode pests including Meloidogyne (Root knot nematode). In a still further embodiment of the present invention the said polynucleotide encodes a protein which is described in International Patent Application Publication Number WO01/00841.

The present invention still further provides a method as described above wherein said explant is obtained from the cotton plant line COKER 312 or COKER 315. The person skilled in the art will appreciate that all cotton plants are applicable to the present invention.

The present invention further provides a method of regenerating a cotton plant from the somatic embryo produced according to the methods described above and a cotton plant obtained by such a method.

The present invention further provides use of a somatic embryo produced according to the methods described above in a method for the production of a cotton plant or a transformed cotton plant.

The present invention still further provides a method for maintaining viable totipotent stomatal cells in culture comprising (a) isolating a totipotent stomatal cell-containing epidermal explant from leaf material of a cotton plant, preferably the leaf material used in this method is obtained as described above; and (b) culturing said explant in a basal medium which comprises between about 2 to about 22 μM NAA and between about 1 to about 5 μM iP; and (c) identifying viable stomatal cells within said explant and maintaining said cells by sub-culturing.

The present invention still further provides the use of a cell according to the preceding paragraph in a method of producing somatic embryos comprising (a) culturing said cell in a basal medium which comprises an embryogenic callus inducing quantity of an auxin and a cytokinin under an embryogenic callus inducing intensity of light until embryogenic callus is formed; and (b) sub-culturing said embryogenic callus onto a somatic embryo differentiation media to produce said somatic embryos. In a further embodiment of the use according to the present invention the auxin is NAA and/or the cytokinin is isopentyladenine (iP).

The present invention still further provides the use as described above wherein the basal medium comprises between about 2 to about 22 μM of NAA and between about 1 to about 5 μM of iP under light irradiation of less than 21 μmol.m⁻².s⁻¹. In a still further embodiment of the use according to the present invention said light irradiation is about 15.8 μmol.m⁻².s⁻¹.

The present invention still further provides the use as described above wherein the said cell is transformed with a polynucleotide prior to induction of embryogenic callus. In a further embodiment of the invention said polynucleotide provides for the production of an agronomic trait selected from the group consisting of herbicide resistance; insect resistance; nematode resistance; fungal resistance; viral resistance; stress tolerance; altered yield; fibre quality and oil quality. In a still further embodiment of the invention said polynucleotide provides for the production of a 5-enolpyruvylshikimate-3-phosphate synthase and/or a crystal endotoxin protein (CRY) and/or a vegetative insecticidal proteins (VIP) and/or for resistance to a herbicide selected from the group consisting of glyphosate; paraquat; acifluorfen; chlorimuron-ethyl; fomesafen; acetochlor; fluazifop-P-butyl; and metolachlor. In a still further embodiment of the invention said polynucleotide provides for resistance to insect pests including Lepidoptera, Spodoptera, Coleoptera, Diptera, Hemiptera, Homoptera, Thysonoptera and/or nematode pests including Meloidogyne (Root knot nematode).). In a still further embodiment of the present invention the said polynucleotide encodes a protein which is described in International Patent Application Publication Number WO01/00841.

Polynucleotides that can be used to transform the cells of the present invention may also be bounded by suitable regulatory elements that are well known to the person skilled in the art. The polynucleotides that can be used to transform the cells of the present invention may also comprise a region that encodes a selectable marker which ultimately allows for selection of the said transformed cell. Suitable selectable markers are well known to the person skilled in the art and include the phosphinothricin acetyl transferase (PAT) gene (U.S. Pat. No. 5,561,236), or neomycin phosphotransferase II (nptII), acetolactate synthase, EPSPS (which confers resistance to glyphosate) genes or the ManA gene which encodes phosphomannose isomerase which provides the plant with the ability to convert mannose-6-phosphate into fructose-6-phosphate. The transformation methods used in accordance with the present invention are also well known to the person skilled in the art and include for example particle mediated biolistic transformation, Agrobacterium-mediated transformation, protoplast transformation (optionally in the presence of polyethylene glycols); sonication of plant tissues, cells in a medium comprising the polynucleotide; micro-insertion of the polynucleotide into totipotent plant material (optionally employing the known silicon carbide “whiskers” technique), electroporation and the like.

Throughout this specification the term stomatal cell(s) includes guard cell(s).

In summary then, the present invention demonstrates inter alia, the feasibility of inducing somatic embryogenesis and plantlet regeneration from callus initiated from stomatal cell complexes using epidermal strips or an epidermal region as a primary explants.

The present invention will now be described by way of the following non-limiting example in combination with Table 1 which illustrates the effect of plant age, bract region and light intensity on callus initiation (%) in epidermal strips of Coker line 312, and on embryogenic callus induction, following three consecutive passages in callus initiation medium. Epidermal strips were cultured on basal medium described previously supplemented with NAA (10.7 μM) and iP (4.9 μM).

### EXAMPLE

Plant Material and Disinfection

Plant material (bracts and young leaves) was collected from plants 3-4 and 9-10 months old, maintained in pots (15 cm in diameter) in greenhouse grown conditions (9±1° C. to 18±1° C., minimum and maximum temperatures, respectively). Preliminary experiments had shown that the epidermis from bracts rather than from young leaves was easier to peel. Therefore, bracts were used. They were collected, from March to June, from various stages of flower development, specifically: green bud stage, opening flower, opened flower, flower exhibiting dead petals and flowers with developing seeds. Explants were surface disinfected by washing in running tap water and immersed in a commercial bleach solution (Domestos™ 15%, v/v), for 15 min and then washed three times in sterile distilled water.

Isolation of Epidermal Strips and Culture

Following disinfection, bracts were soaked in sterile distilled water, for at least 3 h, to facilitate peeling. Epidermal strips were carefully excised from the lower epidermis, using fine forceps under sterile conditions. The enzymatic treatment of epidermal strips was tested for the removal of all contaminating mesophyll and vascular tissues as follows: strips were immersed for 2-3.5 h, in a filter sterilised solution containing Linsmaier & Skoog salts (1965), Cellulysin (0.2% w/v), Hemicellulase (0.2% w/v), polyvinylpyrrolidone (PVP-40, 0.1% w/v) and pH adjusted to 5.5.

Epidermal fragments (3-10 mm size), with or without enzymatic treatment, were placed in Petri dishes (32 mm in diameter) containing 2 ml semi-solid medium, with the cuticle side either in contact with the medium or upwards.

Culture Media and Incubation Conditions

A basal medium containing Murashige and Skoog ((1962) Physiol Plant 15: 473-497.) salts, Trolinder & Goodin ((1988a) Plant Cell Tiss Org Cult 12: 31-42.) vitamins and glucose (166.5 mM equivalent to 30 g.l⁻¹) was used throughout the experiments. For callus initiation, basal medium was supplemented with naphthalene acetic acid (NAA, 2.7, 5.4, 10.7 and 21.5 μM) and isopentenyladenine (iP, 1.3, 2.5 and 4.9 μM). In addition, the effects of thidiazuron (tdz), kinetin (kin) and iP, each at a concentration of 4.9 μM, were tested for their effect on callus morphology and development. Callus initiation was evaluated after 3-6 weeks. Media was used as liquid or solidified (geirite 1.6 g.l⁻¹ plus 7.9 mM MgCl₂). Twenty to thirty epidermal strips were cultured per dish.

The effect of shading of the culture dishes on callus initiation was also evaluated. One or two dishes containing a layer of culture medium (2 ml) were placed on the top of dishes containing the cultures. Dishes were incubated on full light (26.3 μmol. m⁻².s⁻¹) or under reduced light irradiance, respectively, under shade from 1 dish (21.0 μmol. m⁻².s⁻¹) or shading from two dishes (15.8 μmol. m⁻².s⁻¹).

Following callus initiation, calluses were sub-cultured, every 4-5 weeks, to callus initiation medium or to basal medium without growth regulators, either solidified or liquid, agitated (under orbital shaking at 110 rpm) or to the surface of filter paper (Whatman™ n° 1) bridges, inserted into macrowell plates (34 mm in diameter, well), containing 3-4 ml of liquid medium to induce somatic embryogenesis. Liquid cultures were maintained by subculture at 2 week intervals. Primary calli containing embryogenic clumps were sub-cultured to somatic embryo differentiation media. A culture medium similar to embryogenesis callus induction medium was used, but modified to contain 10 mM glutamine (Price and Smith 1979 (Planta 145: 305-307.), Cousins et al. 1991 Aust. J. Plant Physiol. 18: 481-494.), 5.4 μM NAA, 2.5 μM iP and solidified with 0.2% gelrite. The effect of abscisic acid (ABA, 0.0, 0.19, 0.38, 1.9 μM) on somatic embryo histodifferentiation was tested.

Cotyledonary somatic embryos, isolated or in aggregates, were sub-cultured to Stewart and Hsu ((1977) Planta 137: 113-117) medium, without growth regulators (Cousins et al. 1991), containing 55.5 mM glucose. Culture medium was solidified with 0.2% gelrite. Embryos not reaching at least 5 mm in length were re-plated on the same conditions for a further 3-4 weeks. Cultures were incubated on full light (26.3 μMol. m⁻².s⁻¹) for somatic embryo differentiation and growth.

In order to stimulate plantlet development, embryos with a size of 8-10 mm and with a pair of cotyledonary leaves were sub-cultured to a horticultural mix containing a standard potting compost and perlite (1:1). Cultures were grown into Magenta boxes and were incubated at 24 ° C. under 26.3 μmol. m⁻².s⁻¹ light irradiance.

In all media, pH was adjusted to 5.7 with dilute HCl or KOH prior to autoclaving for 15 min at 121° C.

All cultures were maintained under a 16 h light/8 h dark cycle photoperiod, under coolwhite fluorescent tubes, at 30° C., unless otherwise stated.

Stomatal Guard-Cell Viability

Strips were incubated on fluorescein diacetate (FDA) (0.1% w/v prepared in acetone) accordingly to Widholm ((1972) Stain Technol 47: 189-194). Guard cell viability was determined at 3 d and 10 d following epidermal culture. Guard cell duplexes were photographed on Kodak™ EPT 160 T film, using dark-field fluorescence microscopy (LP 520 barrier filter, BG12 exciting filter). The viability of guard cell complexes (mean number of stomatal guard-cells fluorescing per field, out of five counts) and contamination with mesophyll and vascular cell wall fragments were evaluated.

1.0 General Observations

The effects of enzyme treatment of epidermal strips were evaluated in preliminary experiments; a decreased viability of guard cells was observed and, in addition, proved to be detrimental to callus initiation. Therefore, no enzymatic treatment of the epidermal strips was performed in subsequent experiments.

Following the culture of epidermal strips, fluorescein diacetate (FDA) staining at 3 d (as shown in FIG. 2a of Nobre et al) and 10 d (as shown in FIG. 2b of Nobre et al), revealed that only stomatal guard cells survived in culture rather than mesophyll cells. However, stomatal viability within epidermal fragments showed great variation (0-24 guard cells fluorescing/microscopic field) and therefore it was difficult accurately to compare treatments.

2. Callus Initiation

After 3-4 d in culture, guard cell swelling and increased plastid prominence was observed. In a number of guard cell complexes, divisions occurred very early, after 2-3 days in culture, yielding microcolonies after 7 days. Subsequently, callus growth occurred rapidly and a compact callus was produced. However, in other guard cell complexes the first divisions occurred later, usually after 10-14 d, callus growth was slower and microscopic colonies were obtained only after 3 weeks. These calluses were less compact and more friable, under optimised culture medium conditions (see culture medium 2.2).

Following their culture, epidermal strips tended to curl and shrink, loosing contact with the medium. Two to four macroscopic calluses/epidermal strip developed, usually on the periphery of the epidermal strip. Therefore, an improved contact of the guard cells from the periphery may improve their response in culture.

2.1. Origin of the Epidermis

The source of the epidermal tissue, particularly the plant age, the developmental stage of the flower and the bract region from which epidermal strips were obtained were evaluated by assessing callus initiation from epidermal tissues.

2.1.1. The Plant Age

Epidermal strips were excised from the bract base of Coker line 312 and were cultured on medium optimised for this genotype (NAA 10.7+iP 4.9 μM). Two to four independent experiments were carried out. The plant age had a significant effect on callus induction (Table 1). Higher callus induction frequency was observed in guard cell complexes of epidermal strips obtained from older plants (9-10 Months old, 36.3±15.8%) than from younger plants (3-4 Months old, 17.8±10.5%).

2.1.2. Developmental Stage of the Flower

Epidermal strips obtained from whole bracts of Coker line 315 were excised at the following stages of flower development: green bud stage, opening flower, opened flower, flower with dead petals and flower at the stage of seed development (As shown in FIG. 1-A, B, C, D, and E of Nobre et al). Explants were cultured on the medium (NAA 5.4+iP 4.9 μM). Experiments were repeated twice and 385 epidermal strips were used. The developmental stage of the flower was affecting morphogenesis. Callus formation (11.0±1.8%) was observed in epidermal strips obtained from bracts excised from opening flowers, when petals start to become visible. Similar results were obtained in both Coker lines (Coker 312 and 315). Therefore, bracts obtained from opening flowers were used in subsequent experiments.

2.1.3. The Bract Region

Epidermal strips excised from 9-10 months old plants of Coker line 312 were cultured on the medium optimised for this genotype (NAA 10.7+iP 4.9 μM). Two to four independent experiments were carried out. The highest responsive tissue was the basal region of the bract (36.3±15.8%) as compared with explants excised from the top region (13.2±7.1%) (Table 1).

2.2. Culture Medium and Culture Conditions

2.2.1. Culture Medium Composition

Epidermal strips were excised from the whole bract of Coker line 315 and cultured on medium containing NAA (2.7, 5.4, 10.7 and 21.5 μM) and iP (1.3, 2.5 and 4.9 μM). Two to four independent experiments were carried out and 1300 epidermal strips were used in these studies. In general, guard cell viability was observed in all growth regulator combinations. An interesting association was found between the concentrations of NAA of the culture medium and viability of the guard cells: the majority of guard cell complexes exhibited only one guard cell with fluorescence, in the growth regulator combination (10.7 μM NAA+2.5 μM iP). However, the culture of epidermal strips in culture medium containing 21.7 μM NAA and 2.5 μM iP produced a mixture of one and two viable guard cells in the guard cell duplexes, as compared with the growth regulator combination (2.7-5.4 μM NAA+2.5 μM iP) from which both guard cells remained viable.

Callus initiation, growth and morphology from both Coker lines (312 and 315) were influenced by the growth regulators in the callus initiation medium. An improved frequency of callus initiation was obtained on media containing the growth regulator combination (NAA 2.7+iP 4.9 μM). However, these calluses were fast growing, compact and green in colour and failed to re-differentiate into a more friable callus in subsequent subcultures. In addition, there were no statistical significant differences in callus induction frequency within the remaining range of treatments tested.

Concerning callus morphology, in general, the relative frequency of the types of calluses was determined by the relative concentrations of NAA and iP in the culture medium. Lower NAA concentrations and higher cytokinin levels produced fast growing compact green calluses. Increasing the NAA levels (up to 10.7 μM) produced more friable light green calluses, whereas increasing the NAA level further (21.5 μM) gave rise to watery calluses.

Callus growth and development in culture was related to the cytokinin concentration and rapid callus development was obtained on media containing a higher iP concentration (4.9 μM). Macroscopic calluses were obtained in 4-5 weeks on such media conditions. A similar size of callus was obtained in 8 weeks, from media containing a lower iP level (1.2 μM).

The influence of other cytokinins (TdZ and Kin, each at 4.9 μM) was evaluated on embryogenic callus induction in Coker line 312. Culture media were further supplemented with NAA (10.7 μM). Two to four independent experiments were carried out per cytokinin-treatment and 740 epidermal strips excised from the basal region of the bract were used in these experiments. Improved callus induction was observed in both culture media containing iP (36.3±15.8%) and Tdz (31.3±4.5%), but no statistically significant differences were obtained between the frequency of callus initiation (Table 1). In addition, a significant reduction in callus initiation frequency was observed in culture medium containing Kin (8.6±0.7%).

2.2.2. Orientation of the Epidermal Strips

Epidermal strips obtained from the whole bract of Coker line 312 were cultured with cuticle side down or cuticle up on the medium (NAA 10.7+iP 4.9 μM), optimised previously for Coker line 312. Experiments were repeated twice independently and 220 epidermal strips were used. The orientation of the epidermal strips on the culture medium had a significant effect on callus initiation with a higher frequency of callus obtained on epidermal strips which had their cuticles in contact with the culture medium (22.9±10.4%) as compared with those from epidermal strips cultured with the cuticle upwards (7.9±1.9%).

2.2.3. Light Intensity

Epidermal strips were obtained from the basal region of the bract of Coker line 312 and were cultured on medium containing the growth regulator combination (NAA 10.7+iP 4.9 μM). Experiments were repeated 3-4 times. No statistical significant differences were observed on callus initiation from explants cultured in shaded dishes as compared with those plated at full light (Table 1 below). The dark treatment was tested, but no callus initiation was observed from this treatment.

3. Embryogenic Callus Formation and Regeneration

3.1. Somatic Embryogenesis Induction

Following 2-3 subcultures into callus initiation medium (NAA 10.7+iP 4.9 μM), somatic embryogenesis was induced in callus cultures obtained from the culture of epidermal strips isolated from basal bract regions of older plants (9-10 months old) in Coker 312 (Table 1). Embryogenic clumps were observed on the surface of callus cultures. In addition, no embryogenesis occurred on calli obtained on callus initiation media containing other cytokinins rather than iP (Table 1 below). Moreover, no embryogenesis was recorded from calli sub-cultured consecutively to either solidified or liquid culture media without growth regulators.

A factor affecting embryogenesis in Coker 315 was the cytokinin (iP) concentration in the callus initiation medium; embryogenesis was only recorded from calluses initiated on a culture medium containing NAA (10.7-21.4 μM) and iP (1.3 μM) and sub-cultured consecutively to a culture medium containing the growth regulator combination NAA 10.7+iP 4.9 μM.

Light irradiance during callus initiation had an important effect, on subsequent embryogenesis induction, following consecutive subcultures into callus initiation medium. Embryogenesis occurred only in calluses initiated under the lower light irradiance of 15.8 μmol. m⁻².s⁻¹ (Table 1 below).

3.2. Somatic Embryo Differentiation and Plantlet Regeneration

Synchronised embryo differentiation and improved somatic embryo uniformity was observed, after 3-4 weeks, from culture medium supplemented with Abscisic acid (ABA) (0.19-0.38 μM); Embryo differentiation was less uniform from culture medium supplemented with ABA (0.0 or 1.9 μM). After 4-5 weeks in culture, several cotyledonary embryos developed on the surface of the embryogenic callus clump. Somatic embryos, isolated or in aggregates, were sub-cultured to Stewart and Hsu (1977) medium (see above), and further differentiation and somatic embryo growth was observed. Somatic embryos reached a size of 8-10 mm and a small radicule was developing at the end of this stage. Germinated somatic embryos were then transferred to a horticultural substrate. Plantlets were grown to fully mature plants.

TABLE 1

| | Epidermal | Callus | Embryogenic |
|---|---|---|---|
| | strips | initiation | callus/ |
| Parameter tested | cultured | (%) | total calli |
| Plant age and bract region | | | |
| 9-10 Months⁽¹⁾ | | | |
| Base | 337 | 36.3 ± 15.8⁽¹⁾ | 4/61⁽²⁾ |
| Top | 279 | 13.2 ± 7.1⁽¹⁾ | 0/26 |
| 4-5 Months⁽¹⁾ | | | |
| Base | 159 | 17.8 ± 10.5 | 4/28 |
| Top | 55 | 0.0 | 0.0 |
| Culture medium (μM) | | | |
| NAA (10.7) + iP (4.9) | 261 | 36.3 ± 15.8 a | 4/61 |
| NAA (10.7) + TDZ (4.9) | 228 | 31.3 ± 4.5 a | 0/68 |
| NAA (10.7) + KIN (4.9) | 250 | 8.6 ± 0.7 b | 0/21 |
| Light | | | |
| Shade from two dishes | 75⁽³⁾ | 16.0 ± 6.3 a | 5/12 |
| (15.8 μMol. m⁻². s⁻¹) | | | |
| Shade from one dish | 101 | 29.0 ± 13.9 a | 0/25 |
| (21.0 μMol. m⁻². s⁻¹) | | | |
| Full Light | 94 | 31.3 ± 16.2 a | 0/22 |
| (26.3 μMol. m − 2. s − 1) | | | |

⁽¹⁾Mean callus initiation frequency ± SE; means followed by different letters are significantly different at the P = 0.05 level; ⁽²⁾Sum of embryogenic calli regenerated; ⁽³⁾Epidermal strips excised from basal bract regions of younger plants.

1. A method for the production of cotton somatic embryos comprising:
(a) isolating a totipotent stomatal cell-containing epidermal explant from leaf material excised from a cotton plant; and
(b) culturing said explant in a basal medium which comprises an embryogenic callus-inducing quantity of an auxin and a cytokinin under an embryogenic callus inducing intensity of light until embryogenic callus is formed; and
(c) sub-culturing said embryogenic callus onto a somatic embryo differentiation media to produce said somatic embryos.

2. A method according to claim 1 wherein said stomatal cell-containing epidermal explant is from leaf material excised from an area attached to or surrounding an opening flower of a cotton plant.

3. A method according to claim 1 or claim 2 wherein said stomatal cell comprises a guard cell.

4. A method according to any one of claims 1 to 3 wherein the auxin is naphthalene acetic acid (NAA) and/or the said cytokinin is isopentyladenine (iP).

5. A method according to claim 4 wherein said basal medium comprises between about 2 to about 22 μM of NAA and between about 1 to about 5 μM of iP under light irradiation of less than 21 μmol.m⁻².s⁻¹.

6. A method according claim 5 wherein the basal medium comprises about 10.7 μM NAA and about 4.9 μM iP.

7. A method according to claim 5 wherein the basal medium comprises about 10.7 to about 21.4 μM NAA and about 1.3 μM iP.

8. A method according to claim 7 wherein said embryogenic callus is further sub-cultured onto a basal medium comprising about 10.7 μM NAA and about 4.9 μM iP prior to sub-culturing according to step (c).

9. A method according to claim 6 or claim 7 wherein said light irradiation is about 15.8 μmol.m⁻².s⁻¹.

10. A method according to any one of claims 1 to 9 wherein said leaf material comprises a bract or bracteole.

11. A method according to claim 10 wherein said leaf material comprises the base region of said bract or bracteole.

12. A method according to any one of claims 1 to 11 wherein said explant is obtained from a cotton plant that is between about 4 to about 10 months old.

13. A method according to claim 12 wherein the cotton plant is between about 4 to about 5 months old.

14. A method according to claim 12 wherein the cotton plant is between about 9 to about 10 months old.

15. A method according to any one of claims 1 to 14 wherein said leaf material is sterilised prior to production of said explant.

16. A method according to any one of claims 1 to 15 wherein the explant is orientated such that the cuticle of said explant is in contact with said medium.

17. A method according to any one of claims 1 to 16 wherein said somatic embryo differentiation media comprises about 0.15 to about 0.4 μM of abscisic acid (ABA).

18. A method according to claim 17 wherein said somatic embryo differentiation media comprises about 0.19 to about 0.38 μM of ABA.

19. A method according to any one of claims 1 to 18 wherein said cell is transformed with a polynucleotide prior to induction of embroygenic callus.

20. A method according to claim 19 wherein said polynucleotide provides for the production of an agronomic trait selected from the group consisting of: herbicide resistance; insect resistance; nematode resistance; fungal resistance; viral resistance; stress tolerance; altered yield; fibre quality and oil quality.

21. A method according to claim 20 wherein said polynucleotide provides for the production of a 5-enolpyruvylshikimate-3-phosphate synthase and/or a crystal endotoxin protein (CRY) and/or a vegetative insecticidal protein (VIP).

22. A method according to claim 20 wherein said polynucleotide provides for resistance to a herbicide selected from the group consisting of: glyphosate; paraquat; acifluorfen; chlorimuron-ethyl; fomesafen; acetochlor; fluazifop-P-butyl; and metolachlor.

23. A method according to claim 20 wherein said polynucleotide provides for resistance to insect pests including: Lepidoptera, Spodoptera, Coleoptera, Diptera, Hemiptera, Homoptera, Thysonoptera and/or nematode pests including Meloidogyne (Root knot nematode).

24. A method according to any one of claims 1 to 6, 9 to 23 wherein said explant is obtained from a cotton plant line COKER 312.

25. A method according to any one of claims 1 to 5, 7 to 23 wherein said explant is obtained from a cotton plant line COKER 315.

26. A method according to any one of claims 1 to 25 which further comprises regenerating a cotton plant from said somatic embryo.

27. A cotton plant obtained by the method of claim 26.

28. Use of a somatic embryo provided according to any one of claims 1 to 18 in a method for the production of a cotton plant.

29. Use of a somatic embryo provided according to any one of claims 19 to 25 in a method of providing a transformed cotton plant.

30. A method for maintaining viable totipotent stomatal cells in culture comprising:
(a) isolating a totipotent stomatal cell-containing epidermal explant from leaf material excised from a cotton plant; and
(b) culturing said explant in a basal medium which comprises between about 2 to about 22 μM NAA and between about 1 to about 5 μM iP; and
(c) identifying viable cells within said explant and maintaining said cells by sub-culturing.

31. Use of a cell according to claim 30 in a method of producing somatic embryos comprising:
(a) culturing said cell in a basal medium which comprises an embryogenic callus inducing quantity of an auxin and cytokinin and under an embryogenic callus inducing intensity of light until embryogenic callus is formed; and
(b) sub-culturing said embryogenic callus onto a somatic embryo differentiation media to produce said somatic embryos.

32. Use according to claim 31 wherein said auxin is NAA and/or said cytokinin is iP.

33. Use according to claim 32 wherein the basal medium comprises between about 2 to about 22 μM of NAA and between about 1 to about 5 μM of iP under light irradiation of less than 21 μMol.m⁻².s⁻¹.

34. Use according to claim 33 wherein said light irradiation is about 15.8 μMol.m⁻².s⁻¹.

35. Use according to any one of claims 31 to 34 wherein the said cell is transformed with a polynucleotide prior to induction of embryogenic callus.

36. Use according to claim 35 wherein the said polynucleotide provides for the production of an agronomic trait selected from the group consisting of herbicide resistance; insect resistance; nematode resistance; fungal resistance; viral resistance; stress tolerance; altered yield; fibre quality and oil quality.

37. Use according to claim 36 wherein the said polynucleotide provides for the production of a 5-enolpyruvylshikimate-3-phosphate synthase and/or a crystal endotoxin protein (CRY) and/or a vegetative insecticidal proteins (VIP).

38. Use according to claim 35 wherein the said polynucleotide provides for resistance to a herbicide selected from the group consisting of glyphosate; paraquat; acifluorfen; chlorimuron-ethyl; fomesafen; acetochlor; fluazifop-P-butyl; and metolachlor.

39. Use according to claim 35 wherein the said polynucleotide provides for resistance to insect pests including Lepidoptera, Spodoptera, Coleoptera, Diptera, Hemiptera, Homoptera, Thysonoptera and/or nematode pests including Meloidogyne (Root knot nematode).
